(19) United States
(12) Patent Application Publication
(10) Pub. No.: US 2011/0178167 A1
(43) Pub. Date: Jul. 21, 2011

(54) METHOD FOR INHIBITING ACTIVITY AND/OR EXPRESSION OF MATRIX METALLOPROTEINASE, INHIBITING PHOSPHORYLATION OF MITOGEN-ACTIVATED PROTEIN KINASE, AND/OR PROMOTING EXPRESSION OF COLLAGEN USING TERMINALIA CATAPPA LEAF EXTRACT

(76) Inventors: HSIU-MEI CHIANG, Taichung (TW); Kuo-Ching Wen, Taichung (TW)

(21) Appl. No.: 12/838,855

(22) Filed: Jul. 19, 2010

(30) Foreign Application Priority Data
Jan. 21, 2010 (TW) 099101616

(51) Int. Cl. A61K 31/353; A61K 8/97; A61P 17/00; A61P 31/00; A61Q 19/00

(52) U.S. Cl. 514/456

(57) ABSTRACT

A method for inhibiting the activity of matrix metalloproteinase (MMP), inhibiting the expression of matrix metalloproteinase, inhibiting the phosphorylation of mitogen-activated protein kinase (MAPK), and/or promoting the expression of collagen in a mammal is provided, and the method comprises administrating an effective amount of a Terminalia catappa leaf extract to the mammal.

### RELATED APPLICATION

This application claims the benefit of Taiwan Patent Application No. 099101616, filed on Jan. 21, 2010, the disclosure of which is incorporated herein in its entirety by reference.

### BACKGROUND OF THE INVENTION

1. Field of the Invention

The present invention relates to the uses of a Terminalia catappa leaf extract in the inhibition of the activity of matrix metalloproteinase (MMP), inhibition of the expression of matrix metalloproteinase, inhibition of the phosphorylation of mitogen-activated protein kinase (MAPK), and/or promotion of the expression of collagen, especially in the improvement, repair, and/or care of skin.

2. Descriptions of the Related Art

Natural human aging processes include skin flaccidity, wrinkle formation and darkening skin, which gradually appear with aging. The layers of skin from top to bottom are the epidermal layer, basement lamina, and corium laminar. The causes of skin aging can be classified by endogenous and exogenous factors. Endogenous aging is a natural aging process of the human body, including cell apoptosis, hormone decrease, and weakened immunity. The decrease of hormone secretion may slow the metabolism of skin and gradually reduce the production of collagen and elastin because of the deterioration of the function of fibroblasts in the corium laminar. As a result, the connective tissues in the corium laminar degenerate, leading to flaccidity, and even wrinkling of the skin. Furthermore, the degeneration of connective tissues in the corium laminar may decrease the water storage (holding) function of the skin, leading to skin dryness and water deficiency, etc.

Exogenous aging is caused by extrinsic factors, such as sunshine, pollution, free radicals, and smoking. The main factor that damages the skin most and accelerates skin aging is ultraviolet (UV) rays from the sun. Depending on the wavelength, ultraviolet rays can be classified into long wavelength UV (UVA), medium wavelength UV (UVB), and short wavelength UV (UVC). Ultraviolet rays that people are most exposed to in daily life are UVA and UVB, which may cause erythema, sunburns, damage to the deoxyribonucleic acid (DNA) in skin cells, abnormality of the skin immune system, and skin cancer. The aging phenomenon caused by ultraviolet rays is called “photo-aging,” which may lead to an increase of matrix metalloproteinase (MMP) in the corium laminar via the phosphorylation of the mitogen-activated protein kinase (MAPK) pathway. Matrix metalloproteinase may decompose collagen in the skin. Without the support of collagen, skin becomes flaccid, and cuticula may overgrow, leading to darkened skin.

Animal collagen that is currently known can be classified approximately into 21 types. Different kinds of collagen exist in different tissues. Out of all collagen in skin tissues, Type I collagen is the most abundant (80% of skin collagen) and has the most functions. Type III collagen comprises about 20% of the skin collagen. Fibroblasts in subcutaneous tissues mainly produce Type I collagen and Type III collagen for the skin.

As described above, matrix metalloproteinase may decompose collagen and reduce the collagen content in the skin. Accordingly, if the MAPK Pathway or the activity and/or expression of matrix metalloproteinase in cells can be inhibited, the effects of improving/caring for skin quality can be achieved.

It was discovered that the expression of matrix metalloproteinase-1 can be inhibited by ziyuglycoside-I obtained by extracting the roots of Sanguisorba officinalis with 70% ethanol. The expression of matrix metalloproteinase-1 can also be inhibited by sumaflavone and amentoflavone obtained by extracting Selaginella tamariscina with methanol. However, there is still a need to find components that have better effects of inhibiting the activity of matrix metalloproteinase.

The inventors of the present invention found that a Terminalia catappa leaf extract has excellent effects of inhibiting the activity of matrix metalloproteinase, inhibiting the expression of matrix metalloproteinase, inhibiting the phosphorylation of mitogen-activated protein kinase, and/or promoting the expression of collagen, and thus the extract can be used for improving, repairing, and/or caring for the skin.

### SUMMARY OF THE INVENTION

The primary objective of this invention is to provide a method for inhibiting the activity of matrix metalloproteinase (MMP), inhibiting the expression of matrix metalloproteinase, inhibiting the phosphorylation of mitogen-activated protein kinase (MAPK), and/or promoting the expression of collagen in a mammal, comprising administrating an effective amount of a Terminalia catappa leaf extract to the mammal. The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

FIG. 1 is a diagram illustrating the MAPK pathway;

FIG. 2 is a diagram illustrating the mechanism of the Terminalia catappa leaf extract of the present invention;

FIG. 3 is a flow chart illustrating the method for preparing the Terminalia catappa leaf extract of the present invention;

FIG. 4 is a UV-VIS spectrogram of the Terminalia catappa leaf extract of the present invention;

FIG. 5 is a column diagram showing the inhibition rate of the Terminalia catappa leaf extract of the present invention on collagenase;

FIG. 6 is a column diagram showing the inhibition rate of the Terminalia catappa leaf extract of the present invention on collagenase;

FIG. 7 is a protein electrophoresis picture of matrix metalloproteinase (MMP-1, MMP-3, and MMP-9) and Type I pro-collagen in fibroblasts;

FIG. 8 is a protein electrophoresis picture of unphosphorylated and phosphorylated mitogen-activated protein kinases (JNK, ERK, and p38 protein) in fibroblasts;

FIG. 9 is a column diagram showing the survival rate of fibroblasts;

FIG. 10 is a picture showing the skin variation of the rabbits in the primary skin irritation test; and

FIG. 11 is a picture showing eye variation of the rabbits in the eye irritation test.

### DESCRIPTION OF THE PREFERRED EMBODIMENT

The present invention relates to a method for inhibiting the activity of matrix metalloproteinase (MMP), inhibiting the expression of matrix metalloproteinase, inhibiting the phosphorylation of mitogen-activated protein kinase (MAPK), and/or promoting the expression of collagen in a mammal, comprising administrating an effective amount of a Terminalia catappa leaf extract to the mammal. The absorption spectroscopy of the Terminalia catappa leaf extract includes peaks within the following wavelength ranges: from 185 to 225 nm, from 240 to 280 nm, and from 350 to 390 nm. In an embodiment, the absorption spectroscopy of the Terminalia catappa leaf extract includes peaks within the following wavelength ranges: from 195 to 215 nm, from 250 to 270 nm, and from 360 to 380 nm.

The inventors of the present invention discovered that the Terminalia catappa leaf extract of the present invention has the effects of inhibiting the activity of matrix metalloproteinase and inhibiting the expression of matrix metalloproteinase, and may prevent or decrease the destruction of collagen. Matrix metalloproteinase can be classified into collagenase, stromelysin, gelatinase, matrilysin, transmembrane type-MMP, etc. Specifically, the Terminalia catappa leaf extract can effectively inhibit the formation (or expression) of matrix metalloproteinase-1 (MMP-1), matrix metalloproteinase-3 (MMP-3), and matrix metalloproteinase-9 (MMP-9). MMP-1 is also called collagenase-1, which belongs to the collagenase family. Other names for MMP-1 include tissue collagenase or fibroblast-type collagenase. MMP-3 is also called stromelysin-1, which belongs to the stromelysin family, substrates of which include fibronectin, laminin, and non-fibrillar collagen. MMP-9 belongs to the gelatinase family, a major substrate of which is Type IV collagen.

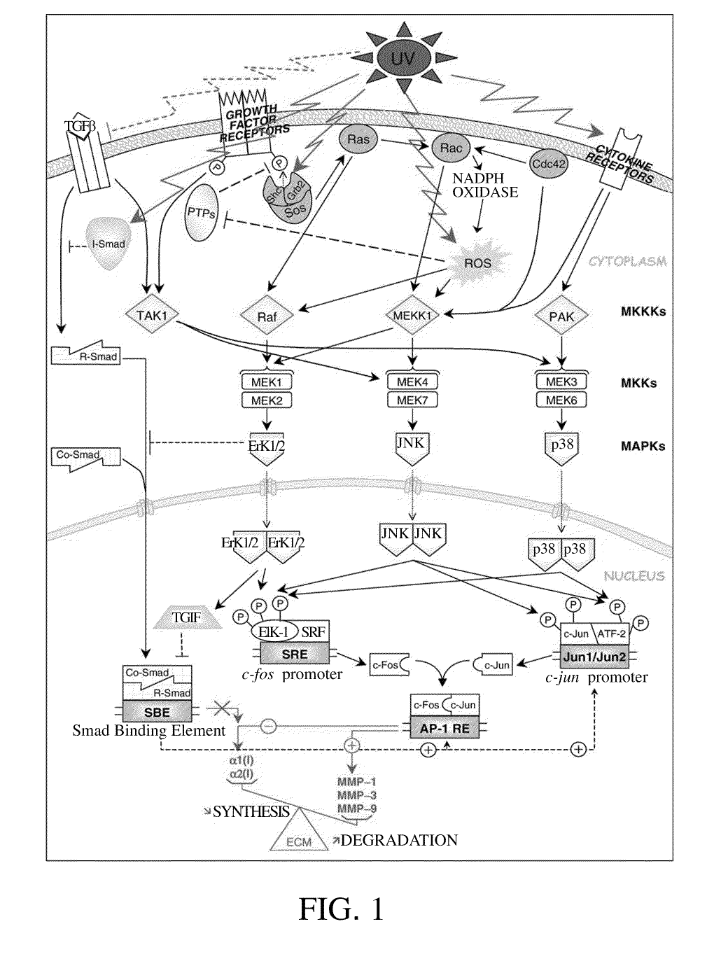

In addition to the effects of inhibiting the activity and/or expression of matrix metalloproteinase, the Terminalia catappa leaf extract also has the effects of inhibiting the phosphorylation of mitogen-activated protein kinase, and especially has the effects of inhibiting the phosphorylation of c-Jun N-terminal Kinase (JNK), extracellular signal-regulated protein kinase (ERK), and p38 protein. As described above, the phosphorylation of mitogen-activated protein kinase may increase the amount of matrix metalloproteinase in corium laminar (the mechanism is shown in FIG. 1, cited from Aging Res. Rev. 2002 September; 1(4): 705-20, which is entirely incorporated hereinto by reference), which further increases the opportunity of the decomposition of collagen, and decreases the collagen content in the skin accordingly.

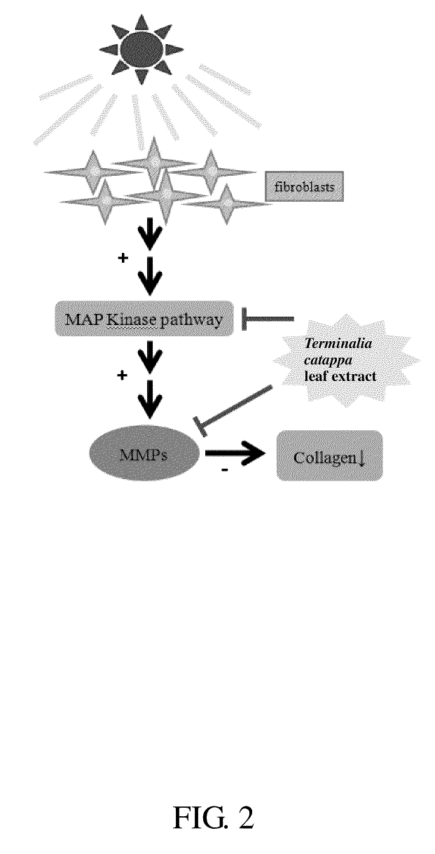

Therefore, as shown in FIG. 2, because the Terminalia catappa leaf extract of the present invention has the effects of (1) directly inhibiting the activity and/or expression of matrix metalloproteinase, and (2) indirectly inhibiting the expression of matrix metalloproteinase by inhibiting the phosphorylation of mitogen-activated protein kinase, the extract may significantly decrease the decomposition of collagen, and thus may effectively improve, repair, and/or care for skin. For example, the extract may has anti-aging and anti-photo-aging properties, thereby reducing skin wrinkling, improving skin quality and skin flaccidity, promoting wound healing, etc.

Furthermore, the Terminalia catappa leaf extract of the present invention further comprises quercetin, which is a flavonoid compound. Not limited by theory, it is believed that the Terminalia catappa leaf extract has the physiological activity of easing skin irritation and anti-aging (see Naz et al, 2007, In vitro antibacterial activity of the extracts derived from Terminalia catappa., Res. J. Microbiol., 2: 180-184, which is entirely incorporated hereinto by reference).

The Terminalia catappa leaf extract of the present invention can be prepared by a method comprising the following steps: a) extracting Terminalia catappa leaves with a polar solvent and collecting the liquid phase; and b) drying the collected liquid optionally. The polar solvent (i.e., extraction solvent) commonly used in this method is selected from a group consisting of water, C₁-C₄ alcohols, and combinations thereof. The polar solvent is preferably selected from a group consisting of water, methanol, ethanol, propanol, butanol, propylene glycol, and combinations thereof. In consideration of the extraction efficiency, the safety of industrial production, product toxicity, etc, water is the most preferred extraction solvent. Besides, the weight ratio of the polar solvent and Terminalia catappa leaves is about 10:1 to about 30:1, preferably about 15:1 to about 25:1, most preferably about 20:1.

In step a), the extraction is carried out for a period to achieve the desired extraction efficiency. For example, when water is used as the extraction solvent, the extraction time is usually at least 30 minutes, more preferably at least 60 minutes, and most preferably at least 90 minutes. The extraction may be optionally assisted with other appropriate extracting approaches (e.g., ultrasonic vibration, heating, etc) to increase extraction efficiency. In addition, the extraction may be optionally repeated one or more times to separate the active components from the inactive components in Terminalia catappa leaves as much as possible, to reduce resource waste and benefit the economy.

In general, depending on the application forms of the Terminalia catappa leaf extract, a drying step may be optionally carried out to dry the Terminalia catappa leaf extract liquid obtained in step a). For example, if the selected extraction solvent is methanol or ethanol, which are not irritating to the skin, and the obtained Terminalia catappa leaf extract liquid is to be applied to the skin directly, additional drying for the extract liquid is not needed. However, if the Terminalia catappa leaf extract of the present invention is to be applied by oral administration, a drying step (such as freeze drying, concentrating under a vacuum condition, and/or ventilation) can be used to remove organic solvents in the Terminalia catappa leaf extract to prevent the organic solvents from harming the body.

In one embodiment of the present invention, Terminalia catappa leaves were extracted with water, heated to acquire an extract liquid, and then freeze dried to dry the extract liquid to acquire a dried Terminalia catappa leaf extract. The extract is soluble in polar solvents (such as solvents selected from a group consisting of methanol, ethanol, propanol, butanol, propanediol, dimethyl sulfoxide, water, and combinations thereof). When 0.125 g of the extract was dissolved in 1 ml water, the pH value of the obtained solution was about 4.5 to about 5.5.

The present invention also relates to a medicament for inhibiting the activity of matrix metalloproteinase, inhibiting the expression of matrix metalloproteinase, inhibiting the phosphorylation of mitogen-activated protein kinase, and/or promoting the expression of collagen, comprising an effective amount of the Terminalia catappa leaf extract of the present invention. Specifically, the Terminalia catappa leaf extract of the present invention can be administrated as a medicament. Based on the effects of inhibiting the activity and/or expression of matrix metalloproteinase, inhibiting the phosphorylation of mitogen-activated protein kinase, and/or promoting the expression of collagen of the medicament of the present invention, the medicament can be particularly used for improving, repairing, and/or caring for the skin.

The medicament of the present invention can be of any suitable form without particular limits. For example, the medicament can be in a form of emulsion, cream, or gel for external use, such as a skin care product, cosmetic, etc. The medicament can also be prepared in the form of food for swallowing or drinking, such as health foods, beauty drinks, etc. Furthermore, the medicament can be of a common pharmaceutical form, such as a tablet, a capsule, a granule, a powder, a fluid extract, a solution, a syrup, a suspension, an emulsion, a tincture, an intravenous injection, a powder injection, a suspension injection, a powder-suspension injection, etc.

The content of the Terminalia catappa leaf extract in the medicament of the present invention may be adjusted according to the age of the treated subject and the purpose of the application (such as reducing skin wrinkles or promoting wound healing), and the usage frequency may also be optionally adjusted. For example, when the Terminalia catappa leaf extract is used for reducing skin wrinkles, the content of the Terminalia catappa leaf extract in the medicament usually ranges from about 0.03 wt % to about 0.4 wt % and preferably ranges from about 0.05 wt % to about 0.25 wt %, based on the total weight of the medicament. The other components and content thereof are dependent on the final form of the medicament. For instance, when the medicament is prepared as a skin care product, any suitable and appropriate amount of emulsion, perfume, and other active components for improving skin quality may be added therein. When the medicament is prepared as a tablet, an appropriate excipient can be used. In general, any component can be added in the medicament, as long as it has no adverse influence on the effects of the Terminalia catappa leaf extract.

The detailed technology and preferred embodiments implemented for the subject invention are described in the following paragraphs accompanying the appended drawings for people skilled in this field to well appreciate the features of the claimed invention.

[Preparation of the Terminalia catappa Leaf Extract]

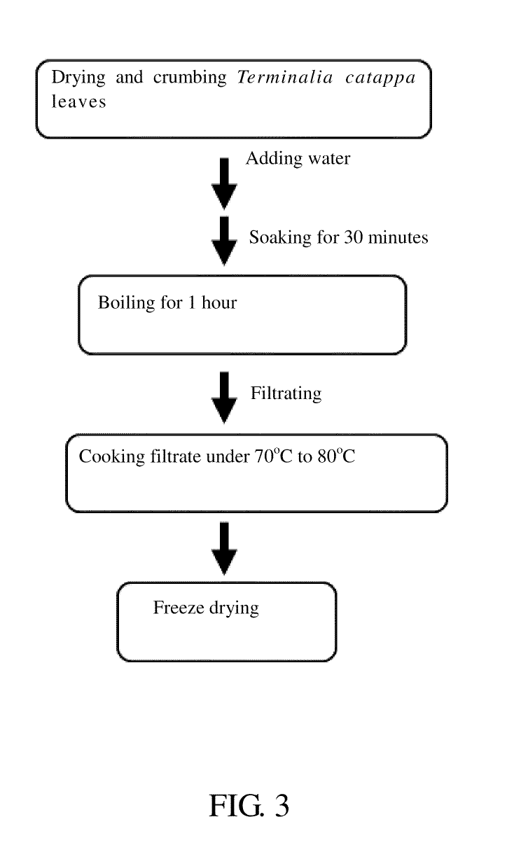

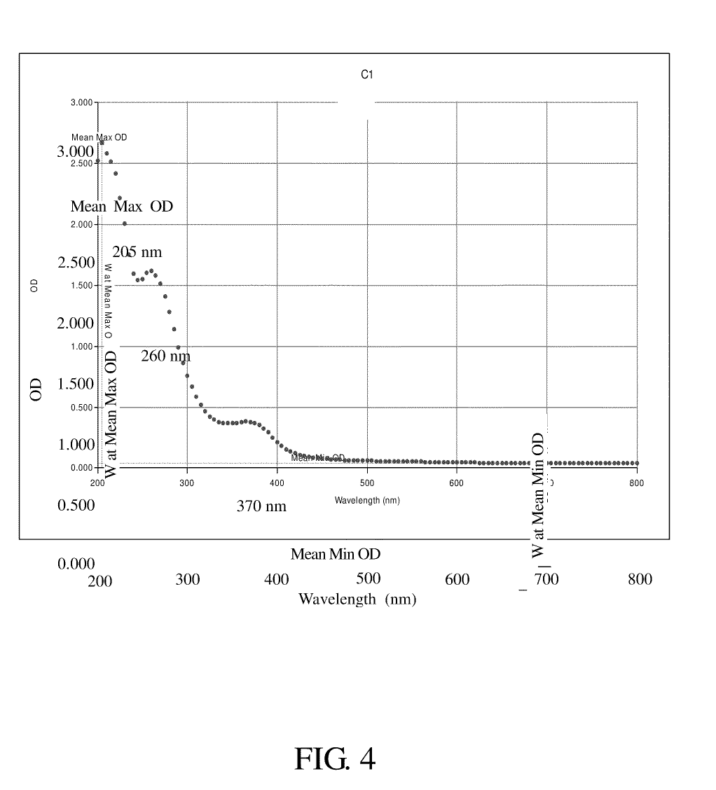

Terminalia catappa leaves in the present invention were from Wu Feng Hsiang, Taichung. FIG. 3 illustrates a flow chart for preparing the Terminalia catappa leaf extract used in the following examples. First, dried Terminalia catappa leaves were crumbled and added into water with a 20-fold weight corresponding to the weight of the leaves. After being soaked for 30 minutes, the leaves were boiled with maximum heating for 1 hour, and filtrated with a Büchner funnel to obtain a filtrate. Then, under 70° C. to 80° C., the filtrate was cooked slowly with minimum heating for 0.5 to 1 hour, and was subjected to a freeze drying to obtain the Terminalia catappa leaf extract of the present invention. An UV-VIS Spectrophotometer (UV-160, Shimadzu) was used to detect the characteristic absorption wavelength of the extract. The UV-VIS absorption spectrogram is shown in FIG. 4.

### Example 1

Experiment A. Inhibition Test of Collagenase Activity

A fluorogenic substrate (fluorogenic peptide substrate I) of collagenase was used in this experiment to evaluate the inhibition effect of the Terminalia catappa leaf extract on collagenase. The collagenase that was used in this experiment was a recombinant collagenase with broad activity. First, water (480 μl) was added into an eppendorf tube, and then 80 μl of a 10-fold diluted buffer solution (prepared by mixing 5 ml of 1M Tris (pH 7.8), 1 ml of 1M CaCl₂, 3.75 ml of 4M NaCl, and 0.25 ml water), 80 μl of the extract (50 to 1,000 μg/ml, in a 50 vol % propanediol aqueous solution), 80 μl collagenase (0.01 mg/ml), and 80 μl of the fluorogenic substrate (fluorogenic polypeptide substrate I, 10 μM) were added into the eppendorf tube. The obtained solution was well mixed, and placed in an incubator under 37° C. for 20 hours. Then, a Luminescence spectrometer (LS50B, PerkinElmer Co. Ltd.) was used to determine the absorption values of the solution under excitation light (328 nm) and radioactive light (393 nm), respectively. Finally, the inhibition rate of the Terminalia catappa leaf extract on collagenase was calculated by the following formula. The experiment was carried out for three times with the same procedure, and average values and standard deviation of the inhibition rate were calculated. The results are shown in Table 1.

$$Inhibition ( \% ) = \frac{( A - B ) \text{?} - ( C - D )}{( A - B )}$$

$$\text{?} \text{indicates text missing or illegible when filed}$$

A: solution comprising collagenase, but not the extract

B: solution comprising no collagenase and the extract

C: solution comprising collagenase and the extract

D: solution comprising no collagenase, but the extract

TABLE 1

| | Group | | | | | |
|---|---|---|---|---|---|---|
| | double | | | | | |
| | deionized | doxycycline | extract | extract | extract | extract |
| | water | (100 μg/ml) | (50 μg/ml) | (100 μg/ml) | (500 μg/ml) | (1,000 μg/ml) |
| Inhibition | −12.26 ± 2.12 | 94.3 ± 0.8 | 81.9 ± 0.7 | 88.1 ± 0.2 | 96.7 ± 0.7 | 100.7 ± 0.7 |
| rate (%) | | | | | | |

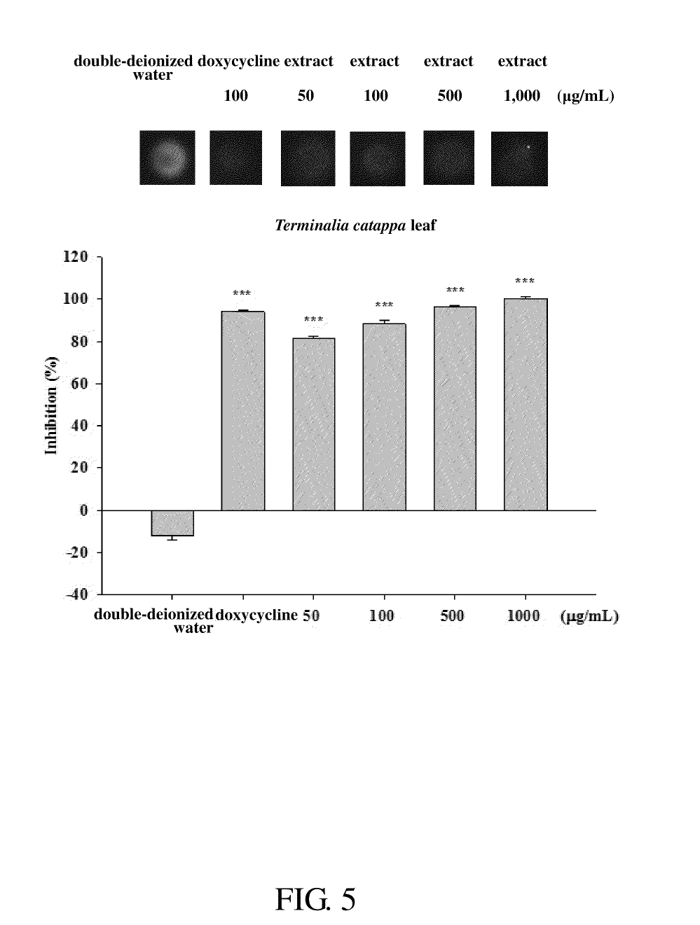

As shown in Table 1 and FIG. 5, the inhibition rate of doxycycline (100 μg/ml) as a positive control is 94.3±0.8%; the inhibition rate of double deionized water as a blank is −12.26±2.12%; the inhibition rate of the Terminalia catappa leaf extract is about 80% to about 100%.

This test shows that the Terminalia catappa leaf extract of the present invention can inhibit the activity of collagenase effectively.

Experiment B. Concentration-Dependent Inhibition Test of Collagenase Activity

To further confirm the inhibition effects of the Terminalia catappa leaf extract on collagenase, the extract was diluted to various concentrations (10 to 500 μg/ml), and the inhibition effect was observed with the same steps in Experiment A.

TABLE 2

| | doxycycline | extract | extract | extract | extract |
|---|---|---|---|---|---|
| Group | (100 μg/ml) | (10 μg/ml) | (50 μg/ml) | (100 μg/ml) | (500 μg/ml) |
| Inhibition rate (%) | 100 ± 0.0 | 82.3 ± 0.9 | 97.7 ± 0.7 | 100.5 ± 0.6 | 101.0 ± 0.4 |

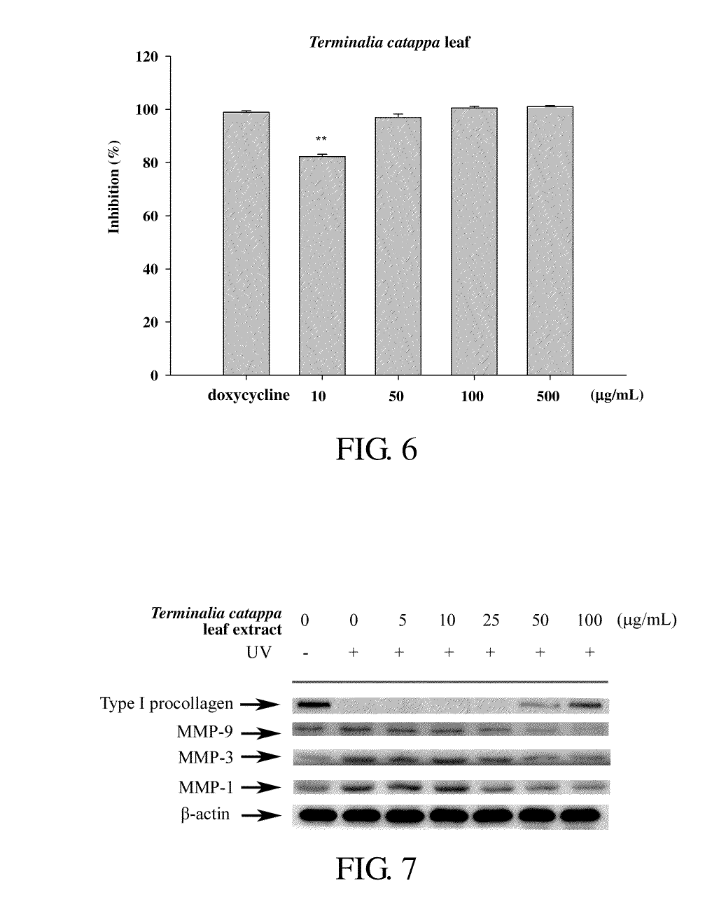

As shown in Table 2 and FIG. 6, above a concentration of 100 μg/ml, the inhibition rate of the extract was greater than 100%. When the extract was diluted to 10 μg/ml, the inhibition rate of the extract was about 80%, and thus, the inhibition effect of the Terminalia catappa leaf extract was concentration-dependent.

### Example 2

Experiment C. Inhibition Test of Matrix Metalloproteinase Expression

First, fibroblasts (human foreskin fibroblast, Bioresource Collection and Research Center (BCRC) number: 60038, purchased from Food Industry Research and Development Institute (FIRDI)) were cultivated in a culture medium (90% Dulbecco's modified Eagle's medium adjusted with 4 mM of L-glutamine, containing 1.5 g/L NaHCO₃, 4.5 g/L glucose, and 10% heat-inactivated fetal bovine serum). After the fibroblasts grew to a density of 80%, the original culture solution was replaced with a culture solution that comprised various concentrations (0 to 100 μg/ml, in dimethyl sulfoxide) of the Terminalia catappa leaf extract, and the fibroblasts were incubated for another 60 minutes. Then, the culture solution was removed, the fibroblasts were rinsed with a phosphate buffer saline (PBS) solution twice, and PBS was added into the culture medium. The fibroblasts were then exposed to 80 mJ/cm² of medium wavelength ultraviolet ray (UVB), and PBS was removed, and a culture solution containing no serum was added into the medium to incubate the fibroblasts. After 24 hours, proteins in the fibroblasts were collected, and a western blotting assay was carried out to observe the expression of matrix metalloproteinase (MMP-1, MMP-3, and MMP-9) and Type I pro-collagen.

As shown in FIG. 7, the expression of matrix metalloproteinase in the fibroblasts increased because of the exposure to UVB, and the expression of MMP-1, MMP-3, and MMP-9 increased 1.5-, 2.2-, and 2.3-fold, respectively. After a treatment of 0.25 μg/ml of the Terminalia catappa leaf extract, the expression of MMP-1 apparently decreased, from 1.5-fold to 1-fold. With a concentration of 50 μg/ml of the extract, the expression of MMP-3 decreased from 2.2-fold to 1.3-fold. Furthermore, with a concentration of 25 μg/ml of the extract, the expression of MMP-9 decreased from 2.3-fold to 1.6-fold. Furthermore, with the increase of the concentration of the extract, the expression of Type I pro-collagen increased remarkably.

This example suggests that the Terminalia catappa leaf extract of the present invention can inhibit the expression of matrix metalloproteinase effectively, and can promote the expression of collagen, and thus, the Terminalia catappa leaf extract can fill the extracellular matrix to make skin elastic and smooth.

### Example 3

Experiment D. Inhibition Test of Mitogen-Activated Protein Kinase Phosphorylation

First, fibroblasts (human foreskin fibroblast, Bioresource Collection and Research Center (BCRC) number: 60038, purchased from Food Industry Research and Development Institute (FIRDI)) were cultivated in a culture medium (90% Dulbecco's modified Eagle's medium adjusted with 4 mM of L-glutamine, containing 1.5 g/L acidic Na₂CO₃, 4.5 g/L glucose, and 10% heat-inactivated fetal bovine serum). After the fibroblasts grew to a density of 80%, the original culture solution was replaced with a culture solution that comprises various concentrations (0 to 100 μg/ml, in dimethyl sulfoxide) of the Terminalia catappa leaf extract, and the fibroblasts were incubated for another 15 minutes. Then, the culture solution was removed, the fibroblasts were rinsed with a phosphate buffer saline (PBS) solution twice, and then PBS was added into the culture medium. The fibroblasts were then exposed to 80 mJ/cm² of UVB, and the PBS was removed. A culture solution containing no serum was added into the medium to incubate the fibroblasts. After 24 hours, proteins in the fibroblasts were collected, and a western blotting assay was carried out to observe the expression of unphosphorylated and phosphorylated mitogen-activated protein kinase (JNK, ERK, and p38 protein) in the fibroblasts.

After fibroblasts were exposed to UVB, the phosphorylation of mitogen-activated protein kinase in the fibroblasts was induced, leading to the activation of mitogen-activated protein kinase pathway (MAPK pathway), further causing photo-aging.

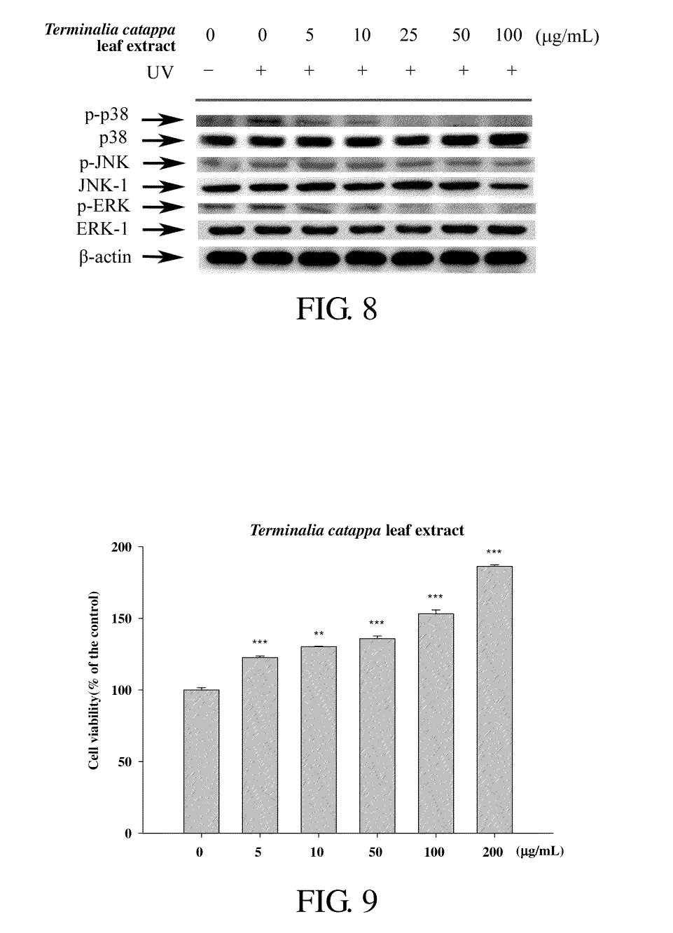

As shown in FIG. 8, after the fibroblasts were exposed to UVB, the expression of phosphorylated ERK, JNK, and p38 was 1.4-, 1.2-, and 1.5-fold compared with the control group without UVB exposure. After treatment with 10 μg/ml of Terminalia catappa leaf extract, the extract showed the inhibiting effect on the phosphorylation of ERK, and the amount of phosphorylated ERK reduced from 1.4-fold to 1.1-fold. With a concentration of 25 μg/ml, the phosphorylation of JNK reduced from 1.2-fold to 1.1-fold Furthermore, with a concentration of 5 μg/ml, the phosphorylation of p38 reduced from 1.5-fold to 1-fold.

This example suggests that the Terminalia catappa leaf extract of the present invention can inhibit the phosphorylation of mitogen-activated protein kinase effectively, and thus, the Terminalia catappa leaf extract can inhibit photo-aging.

### Example 4

Experiment E. Cytotoxicity Test

The cytotoxicity of the Terminalia catappa leaf extract was observed with an MTT (3-(4,5-dimethylthiazol-2-yl)-2,5-diphenyl tetrazolium bromide) assay. First, 50 μl of various concentrations (0 to 200 μg/ml, in a 50 vol % propanediol aqueous solution) of the Terminalia catappa leaf extract was added into a 96-wells culture plate that comprised fibroblasts (10⁴ cells/well). After incubation for 24 hours, 15 μl of a MTT solution (5 mg/ml, in PBS) was added into the well, and then the fibroblasts were placed in an incubator under 37° C. to incubate for another 3 hours. Then, 75 μl of a sodium dodecyl sulfate (SDS) solution (10% SDS, in 0.01N HCl) was added into the culture plate, and the absorbance of each well was measured with a wavelength of 570 nm on the second day. Finally, the cell survival rate was calculated by the following formula, and the cytotoxicity of the extract was observed.

Cell survival rate (%)=absorbance of the experiment group/absorbance of the control group×100

TABLE 3

| | extract concentration (μg/ml) | | | | | |
|---|---|---|---|---|---|---|
| | 0 | 5 | 10 | 50 | 100 | 200 |
| cell survival | 100 ± 1.5 | 122.6 ± 1.2*** | 130.2 ± 0.4** | 135.8 ± 1.8*** | 153.1 ± 2.7*** | 186.2 ± 1.1*** |
| rate (100%) | | | | | | |

*P < 0.01,
***P < 0.001

As shown in Table 3 and FIG. 9, after the fibroblasts were treated with 5 to 200 μg/ml of the Terminalia catappa leaf extract, even under a high concentration of 200 μg/ml, the Terminalia catappa leaf extract showed no cytotoxicity, and the effect of promoting fibroblast growth was observed.

This experiment shows that the Terminalia catappa leaf extract of the present invention is not toxic to cells or the body.

Experiment F. Primary Skin Irritation Test

First, 0.1 g (low dose) and 0.5 g (high dose) of the Terminalia catappa leaf extract were dissolved in 1 ml of a saline solution, respectively. Then, a New Zealand white rabbit (a total of four rabbits was tested in this experiment) was fixed, and the fur on the back of the rabbit was removed. Six squares (each square for 2.5 cm×2.5 cm) for the application of the Terminalia catappa leaf extract were painted with a colored pen on the back of the rabbit. Four parallel lines were made with a sterile needle within one of the squares on the skin of the rabbit to damage the cuticular layer but without bleeding (not to damage the cuticular layer for comparison). The low dose and high dose of the Terminalia catappa leaf extract were applied to the skin of the rabbit within the squares uniformly, respectively. After 24 hours, the skin of the rabbit was wiped with a saline solution to remove the Terminalia catappa leaf extract, and the irritation level of the extract was observed at 24 hours and 72 hours, and a Primary Irritation Index (PII) was acquired. The observation time would be extended if the Terminalia catappa leaf extract irritated the skin of the rabbit. The standards for evaluating the irritation level and Primary Irritation Index are shown in Table 4.

The statistics analysis in this test was based on ANOVA (analysis of variance) and Student's t-test, and if p<0.05, the data is statistically different. Each experiment was carried out for more than three times, and the results of the experiment are represented as a value of mean±standard deviation.

TABLE 4
Standard for scoring Primary Irritation Index

| SKIN REACTIONS | SCORE |
|---|---|
| Erythema and Eschar Formation | |
| No erythema | 0 |
| Very slight erythema (barely perceptible) | 1 |
| Well-defined erythema | 2 |
| Moderate to severe erythema | 3 |
| Severe erythema (beet redness) to slight eschar | 4 |
| formation (injuries in depth) | |
| Edema formation | |
| No edema | 0 |
| Very slight edema (barely perceptible) | 1 |
| Slight edema (edges of area well defined by definite | 2 |
| raising) | |
| Moderate edema (raised approximately 1 mm) | 3 |
| Severe edema (raised more than 1 mm and extending | 4 |
| beyond the area of exposure) | |

Maximum irritation score: 8

| Primary Irritation Index (PII) | Evaluation |
|---|---|
| 0 | No irritation |
| 0.04~0.99 | Irritation barely |
| | perceptible |
| 1.00~1.99 | Slight irritation |
| 2.00~2.99 | Mild irritation |
| 3.00~5.99 | Moderate irritation |
| 6.00~8.00 | Severe irritation |

TABLE 5A
Primary Irritation Index of a low dose (0.1 g) of the Terminalia catappa leaf extract

| | Rabbit No. | | | | | | | | Mean ± standard | |
|---|---|---|---|---|---|---|---|---|---|---|
| Time | 1 | | 2 | | 3 | | 4 | | deviation | |
| (h) | *intact | *abraded | intact | abraded | intact | abraded | intact | abraded | intact | abraded |
| 24 | 0 | 0 | 0 | 0 | 0 | 0 | 0 | 0 | 0.00 ± 0.00 | 0.00 ± 0.00 |
| (Control) | | | | | | | | | | |
| 72 | 0 | 0 | 0 | 0 | 0 | 0 | 0 | 0 | 0.00 ± 0.00 | 0.00 ± 0.00 |
| (Control) | | | | | | | | | | |
| 24 | 0 | 0 | 0 | 0 | 0 | 0 | 0 | 0 | 0.00 ± 0.00 | 0.00 ± 0.00 |
| 72 | 0 | 0 | 0 | 0 | 0 | 0 | 0 | 0 | 0.00 ± 0.00 | 0.00 ± 0.00 |

*intact: evaluation of the irritation to intact skin; abraded: evaluation of the irritation to damaged skin

TABLE 5B
Primary Irritation Index of a high dose (0.5 g) of the Terminalia catappa leaf extract

| | Rabbit No. | | | | | | | | Mean ± standard | |
|---|---|---|---|---|---|---|---|---|---|---|
| Time | 1 | | 2 | | 3 | | 4 | | deviation | |
| (h) | *intact | *abraded | intact | abraded | intact | abraded | intact | abraded | intact | abraded |
| 24 | 0 | 0 | 0 | 0 | 0 | 0 | 0 | 0 | 0.00 ± 0.00 | 0.00 ± 0.00 |
| (Control) | | | | | | | | | | |
| 72 | 0 | 0 | 0 | 0 | 0 | 0 | 0 | 0 | 0.00 ± 0.00 | 0.00 ± 0.00 |
| (Control) | | | | | | | | | | |
| 24 | 0 | 0 | 0 | 0 | 0 | 0 | 0 | 0 | 0.00 ± 0.00 | 0.50 ± 0.50 |
| 72 | 0 | 0 | 0 | 0 | 0 | 0 | 0 | 0 | 0.00 ± 0.00 | 0.50 ± 0.50 |

*intact: evaluation of the irritation to intact skin; abraded: evaluation of the irritation to damaged skin.

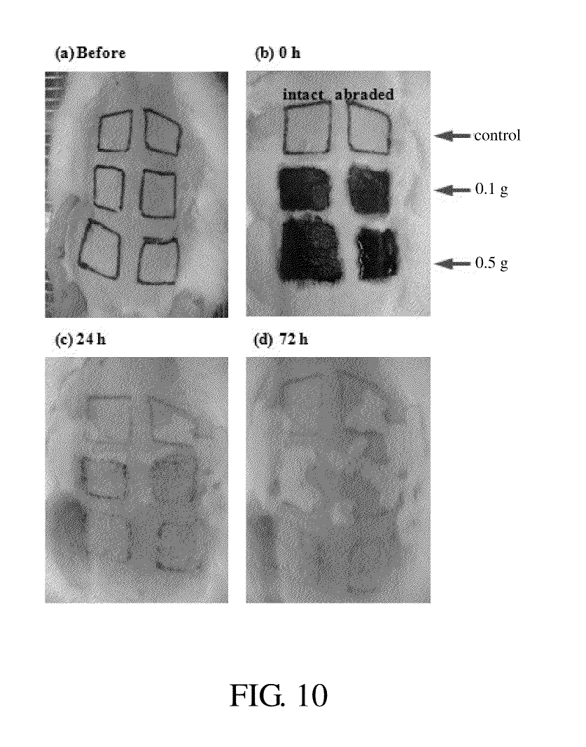

As shown in FIG. 10, Table 5A, and Table 5B, after 24 hours and 72 hours, both the low dose and high dose of the Terminalia catappa leaf extract did not irritate the skin of the rabbits. The irritation index of the extract is 0.0, categorizing the value in the irritation range of “no-irritation.”

This test shows that the Terminalia catappa leaf extract of the present invention is not irritating to the skin.

Experiment G. Eye Irritation Test

After 300 μg of the Terminalia catappa leaf extract was dissolved in 1 ml of a saline solution, the resultant solution was centrifuged at 6,000 rpm for 5 minutes, and the supernatant was collected as a test sample. The sample (600 μg/ml (100 μl) was dropped into the conjunctival sac of one eye of a New Zealand White rabbit (a total of four rabbits was tested in this experiment), whereas the other eye was not administrated with the sample to serve as the control group. The eyes of the rabbit were closed lightly. The influence of the Terminalia catappa leaf extract on the eye of the rabbit was observed at 1, 5, 15, 30 minutes, and 1, 2, 24, 48, 72 hours, etc., and an irritation level was determined. The observation time would be extended if the Terminalia catappa leaf extract generates irritation to the eye of the rabbit. The standards for evaluating the irritation level are shown in Table 6A and Table 6B.

The statistics analysis in this test was based on an ANOVA and Student's t-test, and if p<0.05, the data is significantly different. Each experiment was carried out more than three times, and the results are represented as a value of mean±standard deviation.

TABLE 6A
Standard for scoring eye irritation

| Evaluation | Score |
|---|---|
| CORNEA | |
| A. Opacity | |
| Scattered or diffuse areas of opacity (other than slight dulling of normal | 1 |
| lustre), details of iris clearly visible | |
| Easily discernible translucent area, details of iris slightly obscured | 2 |
| Nacrous area, no details of iris visible, size of pupil barely discernible | 3 |
| Opaque cornea, iris not discernible through the opacity | 4 |
| B. Opaque area of opacity | |
| ≦¼, but >0 | 1 |
| >¼, but </2 | 2 |
| >½, but <¾ | 3 |
| >¾, maximum to the whole area | 4 |
| | Score = A × B × 5 (0 to 80) |
| IRIS | |
| Markedly deepened rugae, congestion, swelling, moderate circumcorneal | 1 |
| hyperemia, or injection, any of these or combination of any thereof, iris | |
| still reacting to light (sluggish reaction is positive) | |
| No reaction to light, hemorrhage, gross destruction (any or all of these) | 2 |
| | Score = A × 5 (0 to 10) |
| CONJUNCTIVA | |
| A. Red level of palpebral conjunctiva | |
| Some blood vessels definitely hyperemic (injected) | 1 |
| Diffuse, crimson color, individual vessels not easily discernible | 2 |
| Diffuse beefy red | 3 |
| B. Conjunctiva edema | |
| Any swelling above normal (includes nictating membranes) | 1 |
| Obvious swelling with partial eversion of the eyelid | 2 |
| Swelling with eyelid about half closed | 3 |
| Swelling with eyelid more than half closed | 4 |
| C. Secretion | |
| Secretion is above normal amount | 1 |
| Secreted and eyelid is wet, eyelash close to eyelid | 2 |
| Secreted and eyelid is wet, most of the area around the eye is wet | 3 |
| | Score = (A + B + C) × 2 (0 to 20) |

Total score = the sum of score of cornea, iris, and conjunctiva

TABLE 6B
Eye irritation level

| Level | Primary Irritation Index (PII) |
|---|---|
| No irritation | 0 |
| Slight irritation | 0.1~15.0 |
| Mild irritation | 15.0~25.0 |
| Moderate irritation | 25.0~50.0 |
| Strong irritation | 50.0~80.0 |
| Severe irritation | 80.0~110 |

TABLE V
Eye irritation score of the Terminalia catappa leaf extract

| | | | | | | Draize scale for |
|---|---|---|---|---|---|---|
| Time | Rabbit No. | | | | mean ± standard | scoring eye |
| (h) | 1 | 2 | 3 | 4 | deviation | irritation |
| 1 | 4 | 0 | 4 | 2 | 2.5 ± 1.9 | 0.6 |
| 24 | 0 | 0 | 0 | 0 | 0.00 ± 0.00 | |
| 48 | 0 | 0 | 0 | 0 | 0.00 ± 0.00 | |
| 72 | 0 | 0 | 0 | 0 | 0.00 ± 0.00 | |

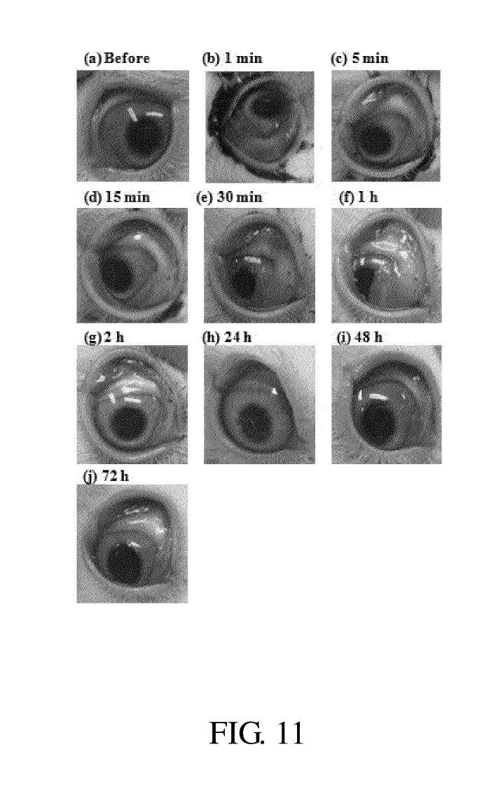

As shown in FIG. 11, in the process of the experiment, the slight congestion of the conjunctiva of three rabbits was observed after 1 minute, and the spherical conjunctiva of the two rabbits swelled above normal. The congestion or swelling of the conjunctiva gradually improved after 30 minutes. After 24 hours, the congestion of the conjunctiva disappeared. As shown in Table 7, the eye irritation score of the Terminalia catappa leaf extract of the present invention was 0.6, belonging to the level of practically “no irritation.”

This test showed that the Terminalia catappa leaf extract of the present invention was not irritating to the eyes.

The above cell model tests and animal safety tests show that the Terminalia catappa leaf extract of the present invention has outstanding effects of inhibiting the activity of matrix metalloproteinase, inhibiting the expression of matrix metalloproteinase, inhibiting the phosphorylation of mitogen-activated protein kinase, and/or promoting the expression of collagen. In addition, the extract is not toxic and irritating, and thus can achieve the effects of improving, repairing, and/or caring for skin without injuring the human body or animal.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. A method for inhibiting the activity of matrix metalloproteinase (MMP), inhibiting the expression of matrix metalloproteinase, inhibiting the phosphorylation of mitogen-activated protein kinase (MAPK), and/or promoting the expression of collagen in a mammal, comprising administrating an effective amount of a Terminalia catappa leaf extract to the mammal.

2. The method as claimed in claim 1, which is for improving, repairing, and/or caring for skin.

3. The method as claimed in claim 1, wherein the absorption spectroscopy of the extract includes peaks within the following wavelength ranges: from 185 to 225 nm, from 240 to 280 nm, and from 350 to 390 nm.

4. The method as claimed in claim 3, wherein the absorption spectroscopy of the extract includes peaks within the following wavelength ranges: from 195 to 215 nm, from 250 to 270 nm, and from 360 to 380 nm.

5. The method as claimed in claim 1, wherein the matrix metalloproteinase comprises matrix metalloproteinase-1 (MMP-1), matrix metalloproteinase-3 (MMP-3), and matrix metalloproteinase-9 (MMP-9), and the mitogen-activated protein kinase comprising c-Jun N-terminal kinase (JNK), extracellular signal-regulated protein kinase (ERK), and p38 protein.

6. The method as claimed in claim 1, wherein the extract comprises quercetin.

7. The method as claimed in claim 1, wherein the extract is prepared by a method comprising the following steps:
a) extracting Terminalia catappa leaves with a polar solvent and collecting the liquid phase; and
b) drying the collected liquid optionally.

8. The method as claimed in claim 7, wherein the polar solvent is selected from a group consisting of water, C₁-C₄ alcohols, and combinations thereof, and the weight ratio of the polar solvent and Terminalia catappa leaves is about 10:1 to about 30:1.

9. The method as claimed in claim 8, wherein the polar solvent is selected from a group consisting of water, methanol, ethanol, propanol, butanol, propylene glycol, and combinations thereof, and the weight ratio of the polar solvent and Terminalia catappa leaves is about 15:1 to about 25:1.

10. The method as claimed in claim 7, comprising repeating step a) one or more times.

11. The method as claimed in claim 1, wherein the extract is administrated as a medicament.
